(19) United States
(12) Patent Application Publication
(10) Pub. No.: US 2012/0029253 A1
(43) Pub. Date: Feb. 2, 2012

(54) LARGE SCALE GREEN MANUFACTURING OF ETHYLENE(ETHENE) USING PLASMA

(76) Inventor: James C. Juranitch, Ft. Lauderdale, FL (US)

(21) Appl. No.: 12/998,694

(22) PCT Filed: Nov. 19, 2009
(86) PCT No.: PCT/US2009/006205

§ 371 Date: Aug. 15, 2011

Related U.S. Application Data
(60) Provisional application No. 61/199,761, filed on Nov. 19, 2008.

(51) Int. Cl. C07C 1/00

(52) U.S. Cl. 585/240; 585/638

(57) ABSTRACT

A method and system for converting waste using plasma into ethylene. The method uses minimal fossil fuel, and therefore produces a minimal carbon footprint when compared to conventional processes. The method includes the steps of supplying a fuel material to a plasma melter; supplying electrical energy to the plasma melter; supplying steam to the plasma melter; extracting a syngas from the plasma melter; extracting hydrogen from the syngas; and forming ethylene from the hydrogen produced in the step of extracting hydrogen.

### RELATIONSHIP TO OTHER APPLICATION

This application claims the benefit of the filing date of U.S. Provisional Patent Application Ser. No. 61/199,761, filed on Nov. 19, 2008, Confirmation No. 5933 (Foreign Filing License granted). The disclosure in the identified provisional patent application is incorporated herein by reference.

### BACKGROUND OF THE INVENTION

1. Field of the Invention

This invention relates generally to methods and systems for extracting hydrogen, and more particularly, to a system for manufacturing ethylene on a large scale.

2. Description of the Related Art

In the current energy environment there is continuing pressure to produce more products and energy in a cost effective and clean way. Fuel prices continue to climb, and emission standards continue to tighten. Most of the modern world has attempted to limit the amount of carbon dioxide that is emitted into the atmosphere. It is considered by many that this gas has some responsibility in the climatic changes commonly referred to as global warming.

The current method of producing Ethylene typically begins with fossil fuels such as light, and heavy hydrocarbons, that are catalytically cracked, or distilled. This process is complex, uses large amounts of energy, and unfortunately puts additional strain on fossil fuel resources as feed stocks that are already limited. The process also continues to liberate significant carbon dioxide and other green house gasses.

In 2005 worldwide production of Ethylene was over 75 million metric tons. This product is a feedstock that is essential in today's world. Ethylene is used in plastics, tires, surfactants, detergents, ethylene glycols, footwear, synthetic lubricants, additives, films, fabrics, sealants, adhesives, tapes, and many other products. As a result of its versatility, ethylene has become an essential building block for modern society. It is a significant disadvantage of current ethylene manufacturing processes that such processes require the consumption of considerable amounts of fuel, and the fossil feed stocks required for such production are directly in conflict with the needs of modern society.

The present invention relates to an energy efficient and modern system and method of producing hydrogen and carbon monoxide, both of which are needed to produce ethylene. The complete process is simple compared to the conventional way of manufacturing ethylene. It also uses renewable materials, and energy, primarily made up of municipal waste as its feed stock. This renewable resource significantly reduces the emissions of carbon based green house gasses that are emitted into the atmosphere.

Plasma melters are now becoming a reliable technology that is used to destroy waste. At this time there are few operational plasma melter installations but the technology is gaining acceptance. It is a characteristic of plasma melters that they produce a low BTU syngas consisting of several different elements. If the plasma melters are operated in a pyrolysis mode of operation, they will generate large amounts of hydrogen and carbon monoxide. The syngas byproduct is typically used to run stationary power generators. The resulting electric power is then sold to the power grid.

It is, therefore, an object of this invention to provide a system for liberating ethylene.

It is another object of this invention to provide a system for liberating ethylene on a large scale and that does not require large electrical generation resources.

It is also an object of this invention to provide a system for liberating ethylene that does not require consumption of natural resources.

It is a further object of this invention to provide a method and system of producing ethylene inexpensively.

It is additionally an object of this invention to provide an inexpensive method of using hydrogen to produce ethylene.

It is yet a further object of this invention to provide an inexpensive method of using a plasma melter to generate large amounts of ethylene.

It is also another object of this invention to provide a method of generating ethylene wherein waste carbon dioxide is obtained from a renewable energy source and therefore does not constitute an addition to the green house gas carbon base.

It is yet another object of this invention to provide a method of using a plasma melter wherein waste carbon dioxide that is generated by the process is destroyed in the plasma melter.

It is a still further object of the invention to convert carbon dioxide into hydrogen and carbon monoxide in a plasma melter with the addition of steam.

### SUMMARY OF THE INVENTION

The foregoing and other objects are achieved by this invention which provides a method of manufacturing ethylene (ethene) on a large scale. In accordance with the invention, the method includes the steps of:

supplying a fuel material to a plasma melter;

supplying electrical energy to the plasma melter;

supplying water to the plasma melter;

extracting a syngas from the plasma melter;

extracting hydrogen from the syngas; and

forming ethylene from the hydrogen produced in the step of extracting hydrogen.

In one embodiment of the invention, the step of supplying water to the plasma melter comprises the step of supplying steam to the plasma melter.

In an advantageous embodiment of the invention, the waste material that is supplied to the plasma melter is a municipal waste. In other embodiments, the waste material is a municipal solid waste, and in still other embodiments the waste material is a biomass. In some embodiments where the waste material is a biomass, the biomass is specifically grown to be supplied to the plasma melter.

In other advantageous embodiments of the invention other waste materials or fuels are employed to achieve the production of ethylene. Such other waste materials or fuels include, for example, fossil fuels. In other embodiments, the fossil fuels are combined to form a fossil fuel cocktail that includes, for example, a biomass material, municipal solid waste, and coal. In still other embodiments, the fossil fuels may be of a low quality, such as brown coal, tar sand, and shale oil.

In one embodiment of the invention, the step of extracting hydrogen from the syngas includes, but is not limited to, the steps of:

subjecting the syngas to a water gas shift process to form a mixture of hydrogen and carbon dioxide; and

extracting hydrogen from the mixture of hydrogen and carbon dioxide.

In a further embodiment, the step of extracting hydrogen from the mixture of hydrogen and carbon dioxide includes, but is not limited to, the step of subjecting the mixture of hydrogen and carbon dioxide mixture to a pressure swing adsorption process. In some embodiments, the step of extracting hydrogen from the mixture of hydrogen and carbon dioxide includes, but is not limited to, the step of subjecting the mixture of hydrogen and carbon dioxide mixture to a molecular sieve. In a further embodiment, the step of extracting hydrogen from the mixture of hydrogen and carbon dioxide includes, but is not limited to, the step of subjecting the mixture of hydrogen and carbon dioxide mixture to an aqueous ethanolamine solution. In yet another embodiment, prior to performing the step of subjecting the syngas to a water gas shift process to form a mixture of hydrogen and carbon dioxide there is provided the step of pre-treating the output of the plasma melter to perform a cleaning and separation of the syngas.

In accordance with an advantageous embodiment of the invention, the step of forming ethylene from the hydrogen produced in the step of extracting hydrogen includes, without limitation, the step of subjecting the hydrogen to a Fischer Tropsch catalytic process. In some embodiments, the Fischer Tropsch catalytic process is an iron-based Fischer Tropsch catalytic process. In one embodiment, prior to performing the step of forming ethylene from the hydrogen produced in the step of extracting hydrogen there is provided the further step of optimizing the production of ethylene by correcting the molar ratio of CO and hydrogen in the Fischer Tropsch catalytic process. The step of correcting the molar ratio of CO and hydrogen in the Fischer Tropsch catalytic process includes, but is not limited to, the step of supplying a mixture of hydrogen and carbon monoxide to the Fischer Tropsch catalytic process.

In an advantageous embodiment of the invention, the step of supplying the mixture of hydrogen and carbon monoxide to the iron-based Fischer Tropsch process includes, but is not limited to, the step of diverting a portion of the hydrogen and carbon monoxide produced by the plasma melter. The step of diverting a portion of the hydrogen and carbon monoxide produced by the plasma melter is performed, in one embodiment, after performing a step of cleaning the hydrogen and carbon monoxide produced by the plasma melter.

In an advantageous embodiment of the invention, there is provided the step of extracting a slag from the plasma melter. In a further embodiment, the step of supplying a waste material to the plasma melter includes, but is not limited to, the step of supplying municipal waste to the plasma melter.

### BRIEF DESCRIPTION OF THE DRAWING

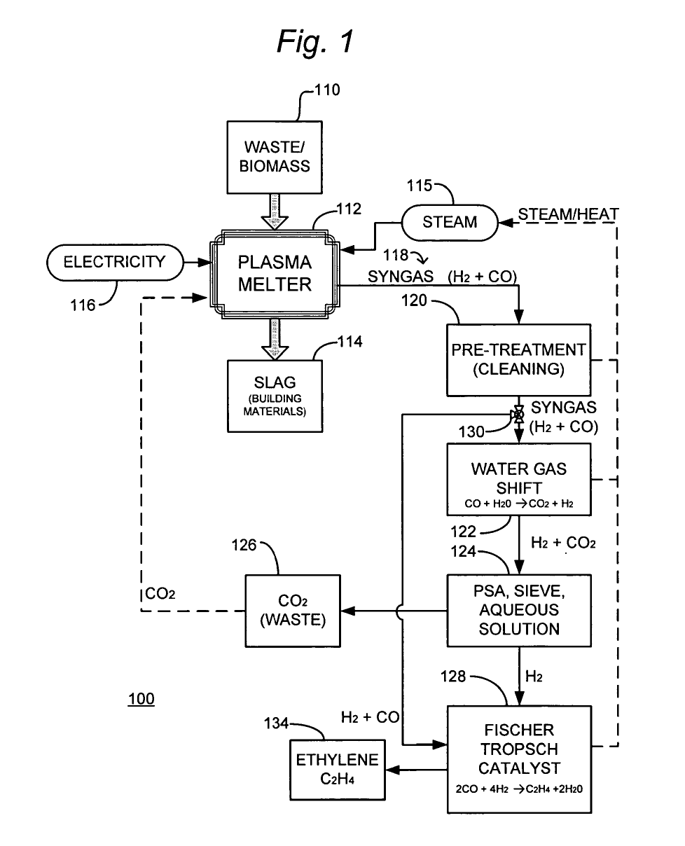

Comprehension of the invention is facilitated by reading the following detailed description, in conjunction with the annexed drawing, in which FIG. 1 is a simplified function block and schematic representation of a specific illustrative embodiment of the invention.

### DETAILED DESCRIPTION

FIG. 1 is a simplified function block and schematic representation of a specific illustrative embodiment of the invention. As shown in this FIGURE, an ethylene producing system 100 receives municipal waste, or specifically grown biomass 110 that is deposited into a plasma melter 112. In the practice of some embodiments of the invention, the process is operated in a pyrolysis mode (i.e., lacking oxygen). Water, which in this specific illustrative embodiment of the invention is used in the form of steam 115, is delivered to plasma melter 112 to facilitate production of hydrogen and plasma. Also, electrical power 116 is delivered to plasma melter 112. A hydrogen rich syngas 118 is produced at an output (not specifically designated) of plasma melter 112, as is a slag 114 that is subsequently removed.

In some applications of the invention, slag 114 is sold as building materials, and may take the form of mineral wool, reclaimed metals, and silicates, such as building blocks. In some embodiments of the invention, the BTU content, plasma production, and slag production can also be “sweetened” by the addition of small amounts of coke or other additives (not shown). Such additives, which may in some embodiments constitute waste materials or fuels include, for example, fossil fuels. In other embodiments, the fossil fuels are combined to form a fossil fuel cocktail that includes, for example, a biomass material, municipal solid waste, and coal. In still other embodiments, the fossil fuels or additives include selectable ones of low quality fuels, such as brown coal, tar sand, and shale oil.

The syngas is cooled and cleaned, and may be separated in certain embodiments of the invention, in a pretreatment step 120. The CO is processed out of the cleaned syngas at the output of a Water Gas Shift reaction 122. The waste carbon dioxide 126 that is later stripped out is not considered an addition to the green house gas carbon base. This is due to the fact it is obtained in its entirety from a reclaimed and renewable source energy. In this embodiment of the invention, the energy source is predominantly municipal waste 110.

In some embodiments, the carbon dioxide is recycled into the plasma melter 112 and reprocessed into CO and hydrogen. A Pressure Swing Adsorption (PSA) process, molecular sieve, aqueous ethanolamine solutions, or other processes are used in process step 124 to separate out carbon dioxide 126. Hydrogen from process step 124 is delivered to a conventional Fischer Tropsch Catalyst process 128, which is a well-known large scale high pressure process for producing ethylene or other carbon-based product, or other similar process, to produce ethylene 134. In some embodiments of the invention, Fischer Tropsch Catalyst process 128 is an iron-based Fischer Tropsch catalytic process.

In this specific illustrative embodiment of the invention, a portion of the CO and hydrogen obtained from pretreatment step 120 is diverted by a flow control valve 130 and supplied to Fischer Tropsch Catalyst process 128. This diverted flow is applied to achieve an appropriate molar ratio of CO and hydrogen, and thereby optimize the production of ethylene.

Pretreatment step 120, Water Gas Shift reaction 122, and Fischer Tropsch Catalyst process 128 generate heat that in some embodiments of the invention is used to supply steam to the plasma melter 112, or to a turbine generator (not shown), or any other process (not shown) that utilizes heat.

Although the invention has been described in terms of specific embodiments and applications, persons skilled in the art may, in light of this teaching, generate additional embodiments without exceeding the scope or departing from the spirit of the invention herein claimed. Accordingly, it is to be understood that the drawing and description in this disclosure are proffered to facilitate comprehension of the invention, and should not be construed to limit the scope thereof.

What is claimed is:

1. A method of manufacturing ethylene on a large scale, the method comprising the steps of:
supplying a fuel material to a plasma melter;
supplying electrical energy to the plasma melter;
supplying water to the plasma melter;
extracting a syngas from the plasma melter;
extracting hydrogen from the syngas; and
forming ethylene from the hydrogen produced in said step of extracting hydrogen.

2. The method of claim 1, wherein said step of supplying water to the plasma melter comprises the step of supplying steam to the plasma melter.

3. The method of claim 1, wherein said step of supplying a fuel material to the plasma melter comprises the step of supplying a fossil fuel to the plasma melter.

4. The method of claim 3, wherein said step of supplying a fuel material to the plasma melter comprises the step of supplying a fuel mixture formed of a selectable combination of a biomass material, municipal solid waste, coal, brown coal, tar sand, and shale to the plasma melter.

5. The method of claim 1, wherein said step of supplying a fuel material to the plasma melter comprises the step of supplying municipal solid waste to the plasma melter.

6. The method of claim 1, wherein said step of supplying a fuel material to the plasma melter comprises the step of supplying a biomass to the plasma melter.

7. The method of claim 6, wherein the biomass is specifically grown for being supplied to a plasma melter.

8. The method of claim 1, wherein said step of extracting hydrogen from the syngas comprises the steps of:
subjecting the syngas to a water gas shift process to form a mixture of hydrogen and carbon dioxide; and
extracting hydrogen from the mixture of hydrogen and carbon dioxide.

9. The method of claim 8, wherein said step of extracting hydrogen from the mixture of hydrogen and carbon dioxide comprises the step of subjecting the mixture of hydrogen and carbon dioxide mixture to a pressure swing adsorption process.

10. The method of claim 8, wherein said step of extracting hydrogen from the mixture of hydrogen and carbon dioxide comprises the step of subjecting the mixture of hydrogen and carbon dioxide mixture to a molecular sieve.

11. The method of claim 8, wherein said step of extracting hydrogen from the mixture of hydrogen and carbon dioxide comprises the step of subjecting the mixture of hydrogen and carbon dioxide to an aqueous ethanolamine solution.

12. The method of claim 8, wherein prior to performing said step of subjecting the syngas to a water gas shift process to form a mixture of hydrogen and carbon dioxide there is provided the step of pre-treating the output of the plasma melter to perform a cleaning of the syngas.

13. The method of claim 8, wherein prior to performing said step of subjecting the syngas to a water gas shift process to form a mixture of hydrogen and carbon dioxide there is provided the step of pre-treating the output of the plasma melter to perform a separation of the syngas.

14. The method of claim 1, wherein said step of forming ethylene from the hydrogen produced in said step of extracting hydrogen comprises the step of subjecting the hydrogen to a Fischer Tropsch catalytic process.

15. The method of claim 14, wherein said step of subjecting the hydrogen to a Fischer Tropsch catalytic process comprises the step of subjecting the hydrogen to an iron-based Fischer Tropsch catalytic process.

16. The method of claim 14, wherein prior to performing said step of forming ethylene from the hydrogen produced in said step of extracting hydrogen there is provided the further step of optimizing the production of ethylene by correcting the molar ratio of carbon monoxide and hydrogen in the Fischer Tropsch catalytic process.

17. The method of claim 16, wherein said step of correcting the molar ratio of carbon monoxide and hydrogen in the Fischer Tropsch catalytic process comprises the step of supplying a mixture of hydrogen and carbon monoxide to the Fischer Tropsch catalytic process.

18. The method of claim 17, wherein said step of supplying the mixture of hydrogen and carbon monoxide to the Fischer Tropsch process comprises the step of diverting a portion of the hydrogen and carbon monoxide produced by the plasma melter.

19. The method of claim 18, wherein said step of diverting a portion of the hydrogen and carbon monoxide produced by the plasma melter is performed after performing a step of cleaning the hydrogen and carbon monoxide produced by the plasma melter.

20. The method of claim 1, wherein there is further provided the step of extracting a slag from the plasma melter.

21. The method of claim 1, wherein the plasma melter is operated in a pyrolysis mode.
